(19) United States
(12) Patent Application Publication
(10) Pub. No.: US 2024/0251813 A1
(43) Pub. Date: Aug. 1, 2024

(54) USE OF LACTASE AND LAC(-) LACTIC ACID BACTERIA (LAB) FOR PRODUCING A FERMENTED MILK PRODUCT

(71) Applicant: Chr. Hansen A/S, Hoersholm (DK)

(72) Inventors: Michael Mitsuo Saito, Valinhos, Sao Paulo (BR); Véronique Jactat, Milwaukee, WI (US); Thomas Janzen, Hoersholm (DK); Mikkel Hviid Danielsen Hornnes, Hoersholm (DK)

(73) Assignee: Chr. Hansen A/S, Hoersholm (DK)

(21) Appl. No.: 18/565,876

(22) PCT Filed: Jun. 10, 2022
(86) PCT No.: PCT/EP2022/065856

§ 371 Date: Nov. 30, 2023

(30) Foreign Application Priority Data
Jun. 11, 2021 (EP) 21178956.5

(51) Int. Cl. A23C 19/032; A23C 9/12; A23C 9/123; A23C 19/068; C12N 1/20; C12R 1/46

(52) U.S. Cl. CPC A23C 19/0328; A23C 9/1206; A23C 9/1238; A23C 19/0684; C12N 1/205; A23V 2400/249; C12R 2001/46

(57) ABSTRACT

The present invention relates to a method for producing a fermented milk product (e.g. a pasta filata cheese) with a relatively stable pH value at the end of the fermentation comprising use of lactase in a first step (a) and lactic acid bacteria (LAB) in a further step, characterized by that the LAB are lactose-deficient (Lac(−)) and capable of metabolizing glucose (Glu(+)). The invention further relates to a Streptococcus thermophilus cell CHCC26980 deposited with registration number DSM32600 and a method to obtain a mutant of this strain.

### FIELD OF THE INVENTION

The present invention relates to a method for producing a fermented milk product (e.g. a pasta filata cheese) with a relatively stable pH value at the end of the fermentation comprising use of lactase and lactic acid bacteria.

### BACKGROUND ART

The food industry uses numerous bacteria, in particular lactic acid bacteria, to improve e.g. the taste and the texture of foods. In the case of the dairy industry, lactic acid bacteria (LAB) are used intensively to bring about the acidification of milk (by fermentation) but also to e.g. texturize the product into which they are incorporated.

Control of post-acidification is of significant commercial relevant importance.

In the art, the term “post-acidification” generally is described as relating to the production of lactic acid by the LAB after the termination of fermentation—for example reads WO2015/193459A1 (Chr. Hansen A/S, Denmark) in paragraph bringing page 1-2:

“Even in methods comprising a rapid cooling step, post-acidification is observed, i.e. the production of lactic acid by the LAB after the termination of fermentation, i.e. after the desired pH has been reached. Post-acidification is considered to represent one of the most important problems during fermentation of milk products today. The further decrease of pH value during processing and storage of the fermented milk product leads to problems with elevated acidity and reduced shelf life.”

WO2015/193459A1 (Chr. Hansen A/S, Denmark) describes different technical solutions for improved control of post-acidification, such as e.g.:
(i): addition of sucrose to milk and use of this sucrose as the only available carbon source for a starter culture comprising lactose-deficient (Lac(−)) Streptococcus thermophilus bacteria (herein termed “ST Lac(−) bacteria”)— see e.g. working Example 2 and 3, where used so-called Acidifix culture comprising ST CHCC17861 Lac(−) strain; or
(ii): a method, wherein the fermentation with a starter culture is carried out in the presence of lactase—see e.g. method “E” of page 23.

In relation to solution (i) discussed above, WO2015/193459A1 reads on page 34-35: “the method of producing a pasta filata cheese of the present invention may comprise the following method steps:
obtaining a milk product with standardized protein, fat and solids;
addition of sucrose sufficient for the acidification culture;
adjusting temperature to coagulation temperature (32-38° C.);
addition of the Acidifix culture (Streptococcus thermophilus CHCC17861 and Lactobacillus delbrueckii subsp. bulgaricus CHCC18944); . . . ”

A pasta filata cheese is a cheese produced by a method comprising a heat treatment step of the curd. The heat treatment step imparts the finished cheese with a fibrous structure and particular stretching properties. Typical pasta filata cheeses include Mozzarella and Provolone, Caciocavallo, Pallone di Gravina, and Scamorza.

A process for making pasta filata cheese may comprise the following steps:
(1) acidifying the milk;
(2) coagulating the resulting acidified milk to form coagulum;
(3) cutting the coagulum to obtain curds and heating the curds to a suitable target temperature;
(4) acidifying the curds to a target level of calcium mineralization to form a product;
(5) heating the product; and
(6) stretching the product.

As known in the art, it is important that the pH value of the “(4) acidifying the curds” step is around important pH 5.0-5.8 for the optimal level of calcium mineralization and thereby the quality/strength of the curd—accordingly, control of post-acidification is of significant commercial importance in relation to e.g. making pasta filata cheese. (See e.g. “Pulari Krishnankutty Nair. “New Trends for Low Moisture Part Skim Mozzarella (Pizza Cheese)”. EC Nutrition 15.3 (2020): 01-05” or “Pasta filata The cheese that melts and stretches; https://www.italianfoodtech.com/the-cheese-that-melts-and-stretches”).

The above discussed Pulari K. Nair article reads:

“Now a day's (sic) addition of citric acid to cheese milk is widely used for making traditional high moisture (e.g. 55% to 60%) Mozzarella cheese”.

One reason for that direct citric acid acidification today is widely used in pasta filata manufacturing procedures relates to post-acidification problems for today used lactic acid bacteria based procedures.

As known in the art—the use of direct acidification in some dairies limits the production of lactic acid bacteria generated aroma compounds and thus the flavor is absent in the final cheese.

The above discussed WO2015/193459A1 (Chr. Hansen A/S, Denmark) reference does not directly and unambiguously describe use of lactase in combination with Lac(−) LAB (e.g. ST Lac(−) bacteria)—it is not disclosed in the description as such and in the Examples is lactase only used in yoghurt Example 5, which does not specify the type of LAB or what they are capable of metabolizing (e.g. Lac(−) or not).

WO2018/130630A1 (Chr. Hansen A/S, Denmark) refers on page 1, lines 20-25 to EPA1-2957180 (in family with above discussed WO2015/193459A1), where it reads: “EP-A1-2 957 180 in one embodiment discloses a method of producing a fermented milk product using a combination of a starter cultures and a conventional lactase”.

The so-called conventional lactase used in the Example 4 of EP-A1-2957180 is HALACTASE™ (Chr. Hansen A/S, Denmark), which is also used in working Examples herein (see below).

WO2018/130630A1 describes use of a so-called low pH stable lactase capable of being active during LAB fermentation, which shall be added either at the start, during or at the end of the fermentation step (see e.g. claim 1)—in working examples the lactase was added at the start of the fermentation together with the starter culture (see e.g. page 30, lines 1-2 of Example 5 and the other working Examples).

Accordingly, WO2018/130630A1 does not directly and unambiguously describe a method, wherein there in step (a) is added lactase to the milk before step (b) of inoculating the milk of step (a) with Lac(−) LAB.

### SUMMARY OF THE INVENTION

The problem to be solved by the present invention is to provide a method for producing a fermented milk product (e.g. a pasta filata cheese) with a relatively stable pH value at the end of the fermentation and wherein an advantage of the produced fermented milk product (e.g. pasta filata cheese) may e.g. be a lower post acidification during e.g. manufacture of the product or storage of the produced fermented milk product.

The solution is based on that the present inventors have identified that it is possible to use lactase in combination with lactose-deficient (Lac(−)) lactic acid bacteria (LAB) in a controlled way, whereby one in commercial relevant scale (use of at least 100 L milk) can improve the control of post-acidification.

Working Examples herein demonstrate improvement of the control of post-acidification by use of Lac(−) Streptococcus thermophilus (herein termed “ST Lac(−) bacteria”) and Lac(−) Lactobacillus delbrueckii.

In view of these positive results for different types of Lac(−) LAB—it is believed that the control of post-acidification novel method/concept as discussed herein would work for substantial all Lac(−) LAB of interest.

Accordingly, a first aspect of the invention relates to a method for producing a fermented milk product comprising following steps:
(a): adding lactase to at least 100 L milk under conditions where the lactase hydrolyses lactose of the milk into glucose and galactose;
(b): inoculating the milk of step (a) with a lactic acid bacteria (LAB) composition comprising from 10⁴ to 10¹⁵ CFU/g viable LAB cells, characterized by that the LAB are lactose-deficient (Lac(−)) and capable of metabolizing glucose (Glu(+)) and optionally also capable of metabolizing galactose (Gal(+));
(c): fermenting the milk with the LAB Lac(−) bacteria of step (b); and
(d): making further adequate steps to finally end up with the produced fermented milk product.

The glucose/galactose generated by the added lactase in step (a) may be seen as the main (if not essentially only) sugar/carbohydrate that the LAB (e.g. ST Lac(−)) of step (b) may use in the fermentation step (c).

Accordingly, the end of the fermentation (alternatively termed termination of the fermentation) may be said to be controlled by the concentration of step (a) lactase generated glucose/galactose in the milk to be fermented in step (c).

As discussed above, the WO2015/193459A1 (Chr. Hansen A/S, Denmark) reference does not directly and unambiguously describe use of lactase in combination with ST lac(−) strains—it is not disclosed in the description as such and in the Examples is lactase only used in yoghurt Example 5, which does not specify anything regarding the type of LAB (e.g. Streptococcus, Lactobacillus or other type of LAB) or even what they are capable of metabolizing (e.g. Lac(−) or not).

Said in other words, the combination of steps (a) and (b) of first aspect above is not directly and unambiguously disclosed in WO2015/193459A1.

In relation to use of lactase—WO2015/193459A1 describes a method, wherein the fermentation with a starter culture is carried out in the presence of lactase—see e.g. method “E” of page 23.

The method of the first aspect is also different (i.e. novel) in relation to this “use of lactase” as such matter, since in step (a) is added lactase to the milk before step (b) of inoculating the milk of step (a) with Lac(−) LAB (e.g. ST Lac(−)).

As discussed above—WO2018/130630A1 (Chr. Hansen A/S, Denmark) also does not directly and unambiguously describe a method, wherein there in step (a) is added lactase to the milk before step (b) of inoculating the milk of step (a) with Lac(−) LAB (e.g. ST Lac(−)).

In working Examples herein are demonstrated—that by adding lactase to milk in accordance with step (a) of the first aspect, it is possible to produce a limited adequate amount of galactose/glucose to limit the activity of Lac(−) LAB (e.g. ST Lac(−)) and thereby to control the growth of the culture to achieve a precise pH of interest.

This technical information is not described or suggested in the art—for instance, it is not described or suggested in above discussed WO2015/193459A1, even though it both describes use of lactase and use of ST Lac(−) bacteria as separate individual possible solutions to the post-acidification problem.

Embodiments of the present invention are described below, by way of examples only.

### DRAWING

FIG. 1: Acidification with the lactose negative culture CHCC17861/CHCC18944 with the addition of glucose and galactose or sucrose. See working Example 1 herein for further details.

FIG. 2: This figure shows that lactase generated glucose/galactose (i.e. step (a) of first aspect herein) were limiting for fermentation with CHCC26980 ST Lac(−) bacteria (i.e. step (c) of first aspect herein) and that acidification level can be controlled by adjusting the lactase generated glucose/galactose concentration. See working Example 2 herein for further details.

FIG. 3: Illustration of an example/embodiment of the invention, wherein the in step (a) lactase hydrolyzed milk is, before step (b) of the first aspect, standardized by addition of standard milk, not treated with lactase, to get a blended milk with a desired glucose/galactose concentration.

FIG. 4: These acidification curves show that the level of acidification with CHCC18944 and CHCC27906 can be controlled according to the available glucose and galactose in the milk, controlled by hydrolysis of part of the milk prior to standardization. See working example 3 herein for further details.

### DETAILED DESCRIPTION OF THE INVENTION

### Deposited Strains/Cells

Above discussed WO2015/193459A1 (Chr. Hansen A/S, Denmark) reads on page 47:

“Lactobacillus delbrueckii ssp. bulgaricus CHCC18944 was deposited with DSMZ-Deutsche Sammlung van Mikroorganismen und Zellkulturen GmbH, Inhoffenstr. 7B, D-38124 Braunschweig, on 2014-06-12 under the accession no. DSM 28910.

Streptococcus thermophilus CHCC17861 was deposited with DSMZ-Deutsche Sammlung van Mikroorganismen und Zellkulturen GmbH, Inhoffenstr. 7B, D-38124 Braunschweig, on 2014-06-12 under the accession no. DSM 28952.”

The deposited strains below are strains that for the first time have been deposited in relation to the present application—i.e. they are novel strains as such.

A sample of the novel Streptococcus thermophilus cell CHCC26980 has been deposited at DSMZ (Deutsche Sammlung von Mikroorganismen und Zellkulturen GmbH, Inhoffenstr. 7B, D-38124 Braunschweig) under the accession number DSM 32600 with a deposit date of 2017 Aug. 22. The deposit has been made under the conditions of the Budapest Treaty on the International Recognition of the Deposit of Microorganisms for the Purposes of patent Procedure.

As discussed in working Examples herein—the herein novel deposited strains have herein relevant advantageous properties.

Accordingly, a separate aspect of the invention relates to a Streptococcus thermophilus cell CHCC26980 deposited with registration number DSM 32600.

A separate aspect of the invention relates to a method to obtain:
a mutant strain of Streptococcus thermophilus cell CHCC26980 deposited with registration number DSM 32600;
   comprising using the deposited strain as starting strain, making mutants of the deposited strain and isolating a novel mutant strain, wherein the mutant strain has retained the property of being ST Lac(−) of the deposited strain.

### Fermented Milk Product

The milk of step (a) of the first aspect and thereby the milk of the fermented milk product the first aspect may e.g. be soy milk or animal milk (such as e.g. goat, buffalo, sheep, horse, camel or cow milk).

Preferably the milk is cow milk.

The fermented milk product is preferably a dairy product such as e.g. yogurt, cheese, kefir or buttermilk.

It may be preferred that the cheese is e.g. fresh cheese product, soft cheese product, cheddar, continental cheese, cottage cheese, pasta filata cheese, pizza cheese or mozzarella cheese.

More preferably, the fermented milk product is a cottage cheese or a pasta filata cheese.

Most preferably, the fermented milk product is a pasta filata cheese, such as e.g. a Mozzarella, a Provolone, a Caciocavallo, a Pallone di Gravina or a Scamorza cheese.

As known in the art—a pasta filata cheese is a cheese produced by a method comprising a heat treatment step of the curd. The heat treatment can be carried out in a number of different ways, including steeping the curds in hot water or whey. In another alternative steam is injected into the curds. The heat treatment step imparts the finished cheese with a fibrous structure and particular stretching properties.

### Adding Lactase to the Milk—Step (a) of First Aspect

Step (a) of the first aspect reads: “adding lactase to at least 100 L milk under conditions where the lactase hydrolyses lactose of the milk into glucose and galactose”.

As known in the art—lactase is an enzyme that is capable of hydrolyzing lactose into glucose and galactose.

In relation to a specific lactase of interest—the skilled person knows under which conditions it is active—i.e. conditions where the lactase hydrolyses lactose of the milk into glucose and galactose.

The art describes numerous different suitable lactases—such as e.g. the HALACTASE™ (Chr. Hansen A/S, Denmark) used in working Examples herein.

It may be preferred that the lactase hydrolysis of step (a) is done for 15 minutes to 4 hours at a temperature from 20 to 45° C.

It may be preferred that the amount of added lactase is from 100 to 20000 NLU/L milk, such as e.g. from 250 to 3000 NLU/L milk.

The neutral lactase units (NLU) is a well known standard unit for the skilled person.

Depending on e.g. the type of milk and fermented milk product of interest—it may be preferred that there in step (a) is hydrolyzed from 0.5 g/L to 60 g/L of lactose, such as e.g. from 3 g/L to 55 g/L of lactose or from 20 g/L to 55 g/L of lactose.

If the milk in step (a) is e.g. virtually completely lactase hydrolyzed, then it may be that the amount of generated glucose/galactose is too high for getting the desired end pH value.

In such a case, the lactase hydrolyzed milk of step (a) may be standardized by adding standard milk, not treated by lactase, to the lactase hydrolyzed milk of step (a).

Accordingly, it may be preferred that the in step (a) lactase hydrolyzed milk is, before step (b) of the first aspect, standardized by addition of standard milk, not treated by lactase, to get a blended milk with a desired glucose/galactose concentration.

In relation to above, it may be preferred that there in step (a) is hydrolyzed 20 g/L to 55 g/L of lactose and the lactase hydrolyzed milk is, before step (b) of the first aspect, subsequently standardized by addition of not lactase treated standard milk to get a blended milk with a desired glucose/galactose concentration—such as e.g. a desired glucose concentration of from 0.5 g/L to 10 g/L, such as from 1 g/L to 10 g/L.

In step (a) may be added one or several fermentable carbohydrates to the milk. The added fermentable carbohydrate is preferably different from lactose, such as e.g. sucrose, glucose or galactose.

Preferably, the lactase is inactivated (by e.g. a heating step such as e.g. a pasteurization step) before the step (b) of the first aspect.

It may be preferred that step (a) of the first aspect relates to adding lactase to at least 200 L milk or at least 1000 L milk.

Inoculatinq the Milk with LAB Lac(−) Bacteria—Step (b) of First Aspect

Step (b) of the first aspect reads:

“(b): inoculating the milk of step (a) with a lactic acid bacteria (LAB) composition comprising from 10⁴ to 10¹⁵ CFU/g viable LAB cells, characterized by that the LAB are lactose-deficient (Lac(−)) and capable of metabolizing glucose (Glu(+)) and optionally also capable of metabolizing galactose (Gal(+));”.

In line with the prior art—the term “lactose deficient” are used in the context of the present invention to characterize lactic acid bacteria (LAB) which have lost the ability to use lactose as a source for cell growth or maintaining cell viability.

Preferably, the lactic acid bacteria (LAB) of step (b) of first aspect is Streptococcus thermophilus (ST), Lactobacillus (preferably Lactobacillus delbrueckii ssp. bulgaricus) and/or Lactococcus (Lactococcus lactis subsp lactis or Lactococcus lactis subsp cremoris).

Preferably, the lactic acid bacteria (LAB) of step (b) of first aspect is Streptococcus thermophilus (ST).

ST Lac(−) bacteria are known to the skilled person and the skilled person may routinely identify/obtain herein suitable ST Lac(−) bacteria (see e.g. above discussed WO2015/193459A1 (Chr. Hansen A/S, Denmark).

Natural/wildtype ST bacteria are capable of metabolizing glucose—accordingly it is evident that it is routine work for the skilled person to obtain/identify herein suitable ST Glu(+) bacteria.

Natural/wildtype ST bacteria are generally not capable of metabolizing galactose.

However, numerous suitable ST Gal(+) bacteria are known to the skilled person—the skilled person may routinely identify/obtain herein suitable ST Gal(+) bacteria (see e.g. above discussed WO2015/193459A1 (Chr. Hansen A/S, Denmark) and WO2019/042881A1 (Chr. Hansen A/S)).

It may herein be preferred that the bacteria cells of step (b) are also capable of metabolizing galactose (Gal(+)).

One reason for this relates to that one may thereby use less amount of lactase to get desired pH value at the end of the fermentation, since LAB Gal(+) (preferably ST Gal(+) bacteria) are also capable of metabolizing the galactose generated by the lactase hydrolysis step (a) of the first aspect.

Another reason is that use of e.g. ST Gal(+) bacteria may reduce the browning in relation to e.g. manufacture of a pasta filate cheese such as e.g. a mozzarella cheese (see e.g. WO2019/042881A1 (Chr. Hansen A/S)—see e.g. working Example 4 herein.

Preferably, in step (b) of the first aspect is the milk inoculated with from 10⁴ to 10¹⁵ cfu (or from 10⁴ to 10¹⁴ cfu) (colony forming units) viable LAB bacteria cells per gram milk, including at least 10⁵ cfu per gram milk, such as at least 10⁶ cfu/g milk, such as at least 10⁷ cfu/g milk, such at least 10⁸ cfu/g milk, such as at least 10⁹ cfu/g milk, such as at least 10¹⁰ cfu/g milk or such as at least 10¹¹ cfu/g milk.

Preferably, the Streptococcus thermophilus (ST) bacteria cell is at least one cell selected from the group consisting of:
(a): a Streptococcus thermophilus cell CHCC17861 deposited with registration number DSM 28952; and
(b): a Streptococcus thermophilus cell CHCC26980 deposited with registration number DSM 32600.

The LAB cells may be a mixture of different LAB strains—such as e.g. a mixture of different ST strains (e.g. a mixture of herein discussed CHCC17861 and CHCC26980)—for instance 10⁸ cfu/g milk of one ST strain (e.g. CHCC17861)+10⁸ cfu/g milk of another ST strain (e.g. CHCC26980), which in sum would imply that the milk is inoculated with 2×10⁸ cfu/g milk viable ST bacteria cells.

Typically, the bacteria (e.g. a starter culture composition) are in a concentrated form including frozen, dried or freeze-dried concentrates.

In step (b) of the first aspect may the milk be inoculated also with other e.g. lactic acid bacteria (LAB) of interest—for instance 10⁴ to 10¹⁵ CFU/g Lactobacillus bacteria cells.

For instance, if there in step (b) is inoculated with from 10⁴ to 10¹⁵ CFU/g LAB Gal(+) cells, then there may of course also be inoculated with other e.g. LAB Gal(−) of interest.

Other LAB of interest should preferably also be lactose-deficient LAB—accordingly, in step (b) is the milk preferably not inoculated with more than 10³ not lactose-deficient bacteria cells, more preferably not inoculated with more than 10² not lactose-deficient bacteria cells and most preferably not inoculated with not lactose-deficient bacteria cells.

It may be preferred (for instance if the fermented milk product is a yogurt) that there in step (b) is also inoculated with from 10⁴ to 10¹⁵ CFU/g of viable lactose-deficient Lactobacillus delbrueckii ssp. bulgaricus—preferably lactose-deficient Lactobacillus delbrueckii ssp. bulgaricus CHCC18944 deposited under the accession no. DSM 28910 (above discussed WO2015/193459A1).

Fermenting the Milk with the Bacteria—Step (c) of First Aspect

Step (c) of the first aspect reads: “fermenting the milk with the LAB Lac(−) bacteria of step (b)”.

The fermenting conditions of step (b) may generally be standard suitable LAB fermentation conditions in relation to a LAB bacterium of interest.

The skilled person knows how to ferment milk with relevant bacteria to make a fermented milk product (e.g. a cheese) of interest—accordingly, there is in the present context no need to describe this in detail.

According to the art and depending on e.g. the ST used, the fermentation temperature may e.g. be from 25° C. to 48° C., such as e.g. from 35° C. to 48° C.

According to the art, the fermentation time in step (b) of the first aspect may be from 2 to 96 hours, such as from 3 to 72 hours or such as from 4 to 48 hours. It may be preferred that the fermentation time in step (b) of the first aspect may be from 2 to 30 hours, such as from 3 to 24 hours.

Preferably, the fermentation of step (c) is done under conditions wherein the fermentation ends with a relatively stable pH value, defined as the pH has not changed more than pH 0.1 during the last 2 hours of the fermentation.

The skilled person knows when one is at the end of the fermentation, which essentially in the present context may be seen as relating to when the pH is not significantly dropping/lowering anymore.

As discussed above, the glucose/galactose generated by the added lactase in step (a) may be seen as the main (if not essentially only) sugar/carbohydrate that the LAB Lac(−) bacteria of step (b) may use in the fermentation step (c).

Accordingly, the end of the fermentation (alternatively termed termination of the fermentation) may be said to be controlled by the concentration of step (a) lactase generated glucose/galactose in the milk to be fermented in step (c).

The pH value of interest at end of the fermentation of step (c) will generally depend on the fermented milk product of interest.

For instance—pH value at end of the fermentation of step (c) may be from pH 3.2 to 6.2, such as e.g. from pH 3.8 to 6.0.

A process for making pasta filata cheese may comprise the following steps:
(1) acidifying the milk;
(2) coagulating the resulting acidified milk to form coagulum;
(3) cutting the coagulum to obtain curds and heating the curds to a suitable target temperature;
(4) acidifying the curds to a target level of calcium mineralization to form a product;
(5) heating the product; and
(6) stretching the product.

As known in the art, it is important that the pH value of the “(4) acidifying the curds” step is around important pH 5.0-5.8 for the optimal level of calcium mineralization and thereby the quality/strength of the curd—accordingly, control of post-acidification is of significant commercial importance in relation to e.g. making pasta filata cheese.

If the fermented milk product of interest is a pasta filata cheese, then during fermenting step (c) of the first aspect involving the acidification of the curd, it is preferred that the pH value at end of the acidification of the curd step is a pH value from pH 5.0 to 5.8.

### Further Adequate Steps to Make Fermented Milk Product of Interest—Step (d) of First Aspect

Step (d) of first aspect relates to making further adequate steps to finally end up with the produced fermented milk product of interest.

As discussed above, the skilled person knows how to make a fermented milk product of interest (e.g. cheese or yogurt)—accordingly, there is no need to describe this in detail in the present context.

### EXAMPLES

### Example 1: Acidification with the Lactose Negative Culture CHCC17861/CHCC18944 in B-Milk with the Addition of Glucose, Galactose and/or Sucrose

### Deposited Strains:

CHCC17861: DSM 28952 ST Lac(−), Glu(+), Gal(+) strain
CHCC18944: DSM 28910 Lactobacillus delbrueckii ssp. bulgaricus Lac(−), Glu(+) Gal(−) strain

As discussed above, CHCC17861 and CHCC18944 were disclosed in WO2015/193459A1 (Chr. Hansen A/S, Denmark).

### Addition of Sugars—Acidification Experiment:

The acidification experiment was set up with the over-night cultures:

CHCC17861 was inoculated in 12 ml M17-1% glucose.

CHCC18944 was inoculated in 10 ml MRS Difco broth.

Incubation occurred over-night at 37° C., anaerobically.

The cultures were then inoculated in 200 ml semi-fat milk (1.5% fat), called B-milk, as follows:
1. 0.8% CHCC17861+0.5% glucose
2. 0.8% CHCC18944+0.5% glucose
3. 0.8% CHCC17861+0.1% CHCC18944
4. 0.8% CHCC17861+0.1% CHCC18944+0.5% sucrose
5. 0.8% CHCC17861+0.1% CHCC18944+0.5% glucose
6. 0.8% CHCC17861+0.1% CHCC18944+0.25% glucose+0.25% galactose
7. B-milk+0.25% glucose+0.25% sucrose+0.25% galactose

The acidification was performed for 48 hours at 41° C. with the CINAC system (Scientific Solutions).

### Results

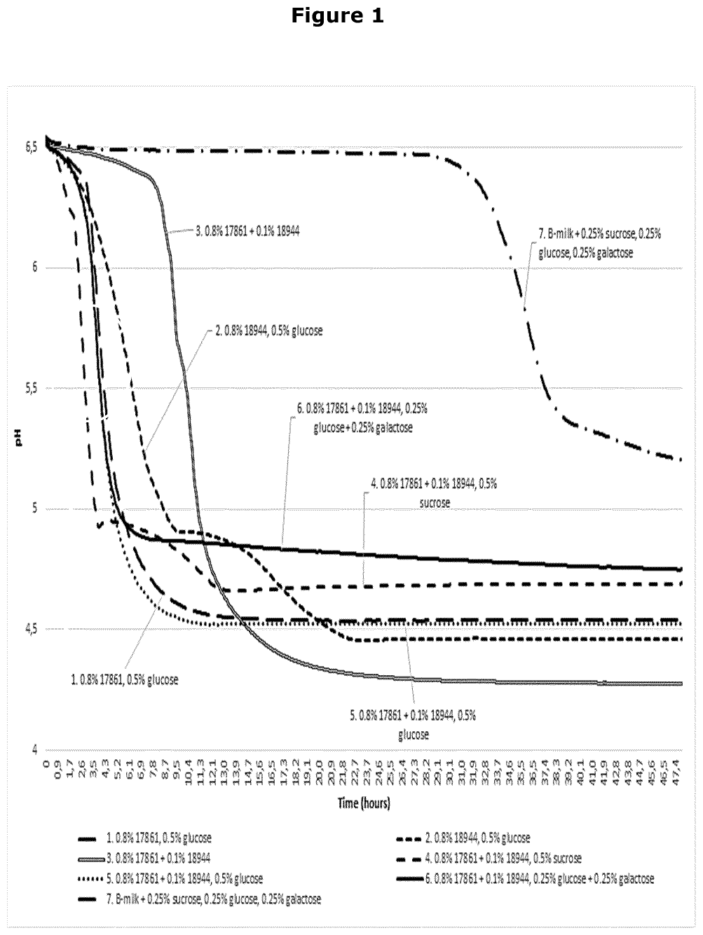

The graph in FIG. 1 shows that the pH can be stabilized after adding 0.5% of a fermentable carbohydrate to the lactose negative culture ST CHCC17861 alone and the CHCC17861/CHCC18944 combination. Initial acidification for the mixed culture is independent whether sucrose, glucose or a mix of glucose and galactose is used. However, the addition of glucose/galactose which resembles the preincubation of milk with lactase, ends at a higher pH as for glucose alone.

### Conclusions

The results demonstrate that the pH can be stabilized after adding 0.5% of a fermentable carbohydrate to the milk, by using the lactose negative culture ST CHCC17861 or the lactose negative culture CHCC18944 alone and the CHCC17861/CHCC18944 combination culture.

### Example 2: Lactase Hydrolysis of Milk Lactose and Acidification with the ST Lac(−) Bacteria Culture ST CHCC26980

### Deposited Strains:

CHCC26980: DSM 32600 ST Lac(−), Glu(+), Gal(−) strain

### Lactose Hydrolysis of Milk Lactose

The lactose content measured in 3 repetitions by using the Lactosens® (a bio-sensor test for the detection of residual lactose in lactose-free milk) was of 4.5%. By using the NOLA fit dosage calculator on pasteurized milk, it has been calculated that 1 liter of skim milk has to be incubated with 5 mL of HA lactase 5200 NLU/g (GIN: 705612, Lot 3488452, density=1.175 g/m) for 1 hour at 30° C. at pH 6.5 and at a lactase dosage of 800 NLU/L to hydrolyze completely the lactose into galactose and glucose.

The residual amount of lactose at the end of incubation was inferior to 0.01% measured with Lactosens®.

Hydrolyzed milk has then been pasteurized (65° C., 30 min) in water bath (counter time was started for 30 min when T° C. of 65° C. was reached) to inactivate the enzyme.

### Standardize 5 Liters of Pasteurized Part Skim Milk to 2.5 g/L of Glucose and Galactose by Using the Lactase Hydrolyzed Milk

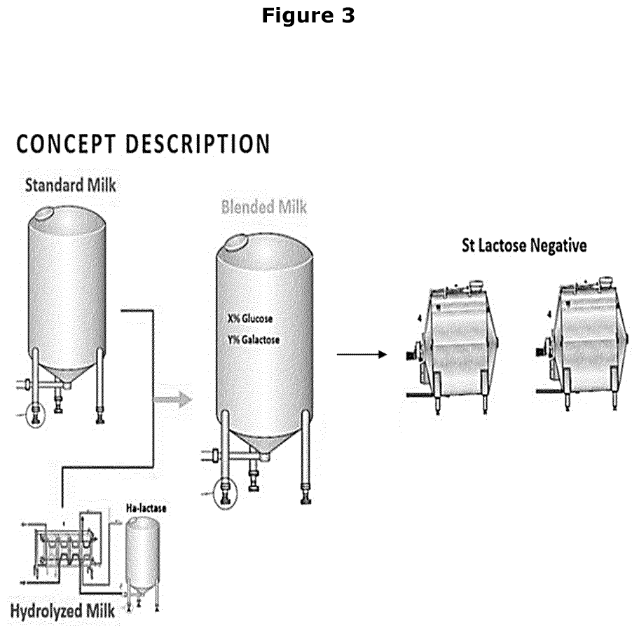

As the lactose content before hydrolysis was of 4.5%, it has been calculated (according to stoichiometric balance) that 2.25% (22.5 g/L) of glucose and an equal amount of galactose were obtained. Based on that calculation, pasteurized part skim milk was standardized by addition of standard (i.e. not lactase treated) milk in order to obtain a 2.5 g/L glucose final content (the same amount of galactose is present—see e.g. FIG. 3 for an illustration).

### Propagation of the Strains:

| Name | Gin | Lot |
|---|---|---|
| F-DVS @STCth26980 (CHCC26980) | 715582 | 3462171 |

Add 1% of sucrose in 1 L of B-Milk
Inoculate with F-DVS of CHCC26980
Incubate overnight at 37° C.
Cool down when pH 4.6
Adjust pH to the initial pH of the standardized milk (used for CINAC) by using a solution of NaOH 0.5M
   Perform Dose Response by Using CHCC26980 after Propagation as Follows:
0.9% 26980
1.8% 26980
2.7% 26980
Milk Control
Test at fixed T° C. 41° C.
Run CINAC for minimum 8 hours or overnight

### Results

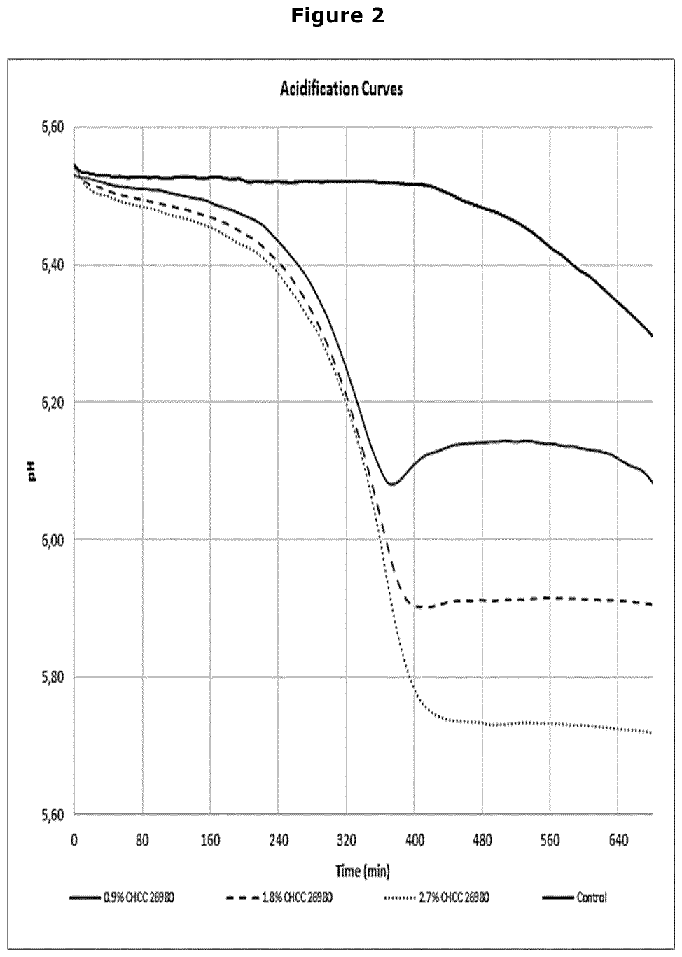

The curves of FIG. 2 show that the end pH of CHCC26980 ST Lac(−) bacteria fermentation is stable for many hours—i.e. there is improved control of post-acidification.

### Conclusions

The results of this Example demonstrate that the lactase generated glucose/galactose (i.e. step (a) of first aspect herein) were limiting for fermentation with CHCC26980 ST Lac(−) bacteria (i.e. step (c) of first aspect herein) and that acidification level can be controlled by adjusting the lactase generated glucose/galactose concentration.

### Example 3: Acidification in Part-Skim Part-Hydrolyzed Milk

Organic part-skim milk was hydrolyzed and standardized as described in Example 2 above.

The hydrolyzed milk was standardized to obtain 0.3% and 0.5% glucose (+equal amount of galactose) respectively.

TABLE 1
The experimental setup for acidification with lac(−) bacteria
in part-hydrolyzed part-skimmed milk.

| | | | Glucose (+gal.) | |
|---|---|---|---|---|
| | Bacterial | | content | |
| | strain | Inoculation | in milk | Temperature |
| 1. | CHCC17861 | 0.6% (w/w) | 0.3% | 41° C. |
| 2. | CHCC18944 | 0.6% (w/w) | 0.3% | 41° C. |
| 3. | CHCC17861 + | 0.53% (w/w) | 0.3% | 41° C. |
| | CHCC18944 | 0.07% (w/w) | | |
| 4. | CHCC27906 | 0.4% (w/w) | 0.5% | 41° C. |

Acidification was done for 18 hours as described in Table 1.

### Results

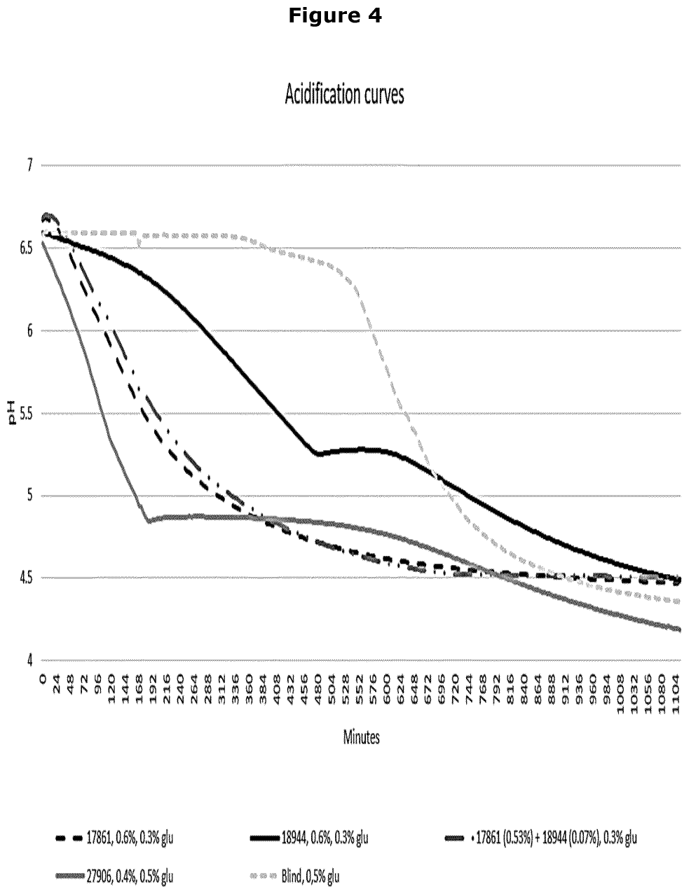

The results are shown in FIG. 4 and show a stop in the acidification for around 2 hours for CHCC18944 and a stop in the acidification for around 6 hours for CHCC27906.

### Conclusion

The curves of CHCC18944 and CHCC27906 show that by reducing the amount of glucose and galactose to specific levels, a break or stop in the fermentation can be obtained. This characteristic is of high potential value in a pasta filata production process, in order to avoid a pH lower than the limits of the specific process, typically 5.0-5.2 in a traditional process. The acidification stop would also be of value in a cottage cheese production process, where it is an advantage to avoid post-acidification, so here strain CHCC27906 ST Lac(−) could be beneficial to use. The exact pH of temporary stabilization can be adjusted by changing the level of glucose and galactose in the milk, thus the acidification can be custom tailored for different cheese types, such as pasta filata or cottage cheese, where stabilization at different pH values is required.

### Example 4: Carbohydrate Analysis

The acidified milk cultures from example 3 were analysed for the concentration of the carbohydrates glucose, galactose, and lactose at the end of the fermentation.

For this, the mono- and disaccharides were analysed by high-performance anion-exchange chromatography with pulsed amperometric detection (HPAE-PAD), on a Dionex ICS-5000, ICS-6000 or Integrion system (Thermo Fischer Scientific, Waltham, MA, USA). The systems were all equipped with a Dionex™ CarboPac™ PA210 column (4 mm×250 mm, 4 μm), and a EGC KOH Eluent Generator Cartridge.

The results are indicated in Table 2.

TABLE 2
Results from carbohydrate analysis in lactase treated
and fermented milk. Results are shown in mg/g

| Unique | | | | |
|---|---|---|---|---|
| Sample | | Glucose | Galactose | Lactose |
| ID | Culture | mg/g | mg/g | mg/g |
| 1 | 17861, 0.6% inoculation in 0.3% glucose hydrolysed milk | 0.51 | 0.33 | 39.25 |
| 2 | 17861, 0.6% inoculation in 0.3% glucose hydrolysed milk | 0.55 | 0.37 | 39.71 |
| 3 | 17861, 0.6% inoculation in 0.3% glucose hydrolysed milk | 0.52 | 0.33 | 39.58 |
| | Average 1-3 | 0.53 | 0.34 | 39.51 |
| 4 | 18944, 0.6% inoculation in 0.3% glucose hydrolysed milk | <LOD | 6.45 | 34.68 |
| | | (0.08) | | |
| 5 | 18944, 0.6% inoculation in 0.3% glucose hydrolysed milk | <LOD | 6.47 | 35.3 |
| | | (0.08) | | |
| 6 | 18944, 0.6% inoculation in 0.3% glucose hydrolysed milk | <LOD | 6.83 | 37.47 |
| | | (0.08) | | |
| | Average 4-6 | | 6.58 | 35.82 |
| 7 | 17861 (0.53%) + 18944 (0.07%) inoculation in 0.3% | <LOD | 0.43 | 41.04 |
| | glucose hydrolysed milk | (0.08) | | |
| 8 | 17861 (0.53%) + 18944 (0.07%) inoculation in 0.3% | <LOD | 0.46 | 39.82 |
| | glucose hydrolysed milk | (0.08) | | |
| 9 | 17861 (0.53%) + 18944 (0.07%) inoculation in 0.3% | <LOD | 0.49 | 39.81 |
| | glucose hydrolysed milk | (0.08) | | |
| | Average 7-9 | | 0.5 | 40.2 |
| 10 | 27906, 0.4% inoculation in 0.5% glucose hydrolysed milk | <LOD | 2.2 | 35.94 |
| | | (0.08) | | |
| 11 | 27906, 0.4% inoculation in 0.5% glucose hydrolysed milk | <LOD | 2.09 | 36.83 |
| | | (0.08) | | |
| 12 | 27906, 0.4% inoculation in 0.5% glucose hydrolysed milk | <LOD | 2.3 | 36.39 |
| | | (0.08) | | |
| | Average 10-12 | | 2.20 | 36.39 |
| 13 | Blind 0.5% hydrolysed milk | 3.23 | 8.73 | 25.71 |

The percentage reduction of galactose compared with the non-inoculated bottle (13) is indicated in Table 3. The vast amount of galactose (>90%) is fermented when the galactose-positive strain CHCC17861 or the culture CHCC17861 plus CHCC18944 is used. The carbohydrate data for 0.3% hydrolysed milk are missing. Even if we postulate that the original galactose level for the 0.3% milk, which was used for the fermentation of CHCC17861 as single strain, was lower, then it can still be concluded that the major part of the galactose was fermented.

This would lead to the explained advantages of a significantly reduced galactose concentration in the final product which can reduce the level of browning of pizza cheese. Additional advantages would be a lower risk for growth of contaminants growing on elevated concentrations of galactose, and also avoidance of post acidification due to the activity of those contaminants (in addition to the activity of the starter culture), and also a higher quality of whey due to reduction of stickiness of whey, assigned to high galactose levels.

TABLE 3
Percentage reduction of galactose compared to
the non-inoculated sample of hydrolysed milk.

| | Galac- | |
|---|---|---|
| | tose | Galactose |
| Sample | mg/g | reduction % |
| Blind 0.5% hydrolysed milk | 8.73 | 0 |
| 17861, 0.6% inoculation in 0.3% hydrolysed milk | 0.34 | 96 |
| 18944, 0.6% inoculation in 0.3% hydrolysed milk | 6.58 | 25 |
| 17861 (8/9) + 18944 (1/9), 0.6% inoculation | 0.50 | 94 |
| in 0.3% hydrolysed milk | | |
| 27906, 0.4% inoculation in 0.5% hydrolysed milk | 2.2 | 75 |

### REFERENCES

1. WO2015/193459A1 (Chr. Hansen A/S, Denmark)
2: Pulari Krishnankutty Nair. “New Trends for Low Moisture Part Skim Mozzarella (Pizza Cheese)”. EC Nutrition 15.3 (2020): 01-05″ or
3: Pasta filata The cheese that melts and stretches; https://www.italianfoodtech.com/the-cheese-that-melts-and-stretches/
4: WO2018/130630A1 (Chr. Hansen A/S, Denmark)
5: WO2019/042881A1 (Chr. Hansen A/S, Denmark)

1. A method for producing a fermented milk product comprising:
(a) adding lactase to at least 100 L milk under conditions where the lactase hydrolyses lactose present in the milk into glucose and galactose;
(b) thereafter, inoculating the milk obtained at step (a) with a lactic acid bacteria (LAB) composition comprising from 10⁴ to 10¹⁵ Colony Forming Units/g (CFU/g) of viable LAB cells, wherein the LAB are lactose-deficient (Lac(−)) and capable of metabolizing glucose (Glu(+)), optionally wherein the Lac(−)Glu(+) LAB are capable of metabolizing galactose (Gal(+));
(c) fermenting the milk with the Lac(−)Glu(+) LAB; and
(d) further processing the fermented milk to obtain the fermented milk product.

2. The method of claim 1, wherein the Lac(−)Glu(+) LAB comprises LAB of one or more species selected from Streptococcus thermophilus (ST), Lactobacillus, and Lactococcus.

3. The method of claim 1, wherein the Lac(−)Glu(+) LAB comprises LAB of species Streptococcus thermophilus (ST).

4. The method of claim 1, wherein the milk of step (a) is cow milk and the fermented milk product obtained at step (d) is yogurt, cheese, kefir, or buttermilk.

5. The method of claim 4, wherein the fermented milk product obtained at step (d) is a cottage cheese or a pasta filata cheese.

6. The method of claim 1, wherein the amount of lactase added in step (a) is from 250 to 20000 NLU/L milk and wherein the lactase hydrolyzes from 0.5 g/L to 60 g/L of lactose.

7. The method of claim 1, further comprising inactivating the lactase after step (a) and before step (b).

8. The method of claim 1, wherein the Lac(−)Glu(+) LAB are capable of metabolizing galactose (Gal(+)).

9. The method of claim 1, wherein in step (b) the milk is not inoculated with more than 10³ bacteria cells that are not lactose-deficient.

10. The method of claim 1, wherein in step (b) the milk is not inoculated with more than 10² bacteria cells that are not lactose-deficient.

11. The method of claim 1, wherein in step (b) the milk is not inoculated with bacteria cells that are not lactose-deficient.

12. The method of claim 9, wherein in step (a) the lactase hydrolyzes from 0.5 g/L to 60 g/L of lactose.

13. The method of claim 9, wherein in step (a) the lactase hydrolyzes from 20 g/L to 55 g/L of lactose.

14. (canceled)

15. (canceled)

16. (canceled)

17. The method of claim 12, wherein the amount of lactase added in step (a) is from 250 to 20000 NLU/L milk.

18. The method of claim 1, wherein the fermentation of step (c) ends with a relatively stable pH, defined as the pH has not changed more than 0.1 pH units during the last 2 hours of the fermentation.

19. The method of claim 1, wherein at the end of the fermentation of step (c) the fermented milk has a pH of from 3.2 to 6.2.

20. The method of claim 1, wherein the fermented milk product obtained at step (d) is a pasta filata cheese and wherein fermenting step (c) comprises a step of acidifying curds, wherein at the end of the acidifying curds step the fermented milk has a pH of from 5.0 to 5.8.

21. (canceled)

22. The method claim 3, wherein the Lac(−) Glu(±) LAB of species Streptococcus thermophilus (ST) comprise cells of one or more strains selected from:
(a) Streptococcus thermophilus strain CHCC17861 deposited with DSMZ-Deutsche Sammlung van Mikroorganismen und Zellkulturen GmbH, Inhoffenstr. 7B, D-38124 Braunschweig (DSMZ) under Accession Number DSM 28952; and
(b) Streptococcus thermophilus strain CHCC26980 deposited with DSMZ under Accession Number DSM 32600.

23. A cell of Streptococcus thermophilus strain CHCC26980 deposited with DSMZ under Accession Number DSM 32600.

24. A method for obtaining a mutant Lac(−)Glu(+) strain of Streptococcus thermophilus, comprising making mutants of strain CHCC26980 deposited with DSMZ under Accession Number DSM 32600, and selecting a mutant that is Lac(−).
